(19) United States
(12) Patent Application Publication
(10) Pub. No.: US 2014/0081251 A1
(43) Pub. Date: Mar. 20, 2014

(54) METHOD AND APARATUS FOR SKIN REDUCTION

(71) Applicant: Joseph Giovannoli, Park Ridge, NJ (US)

(72) Inventor: Joseph Giovannoli, Park Ridge, NJ (US)

(21) Appl. No.: 14/026,879

(22) Filed: Sep. 13, 2013

Related U.S. Application Data
(63) Continuation-in-part of application No. 11/175,004, filed on Jul. 5, 2005, now Pat. No. 8,535,299.
Application No. 11/175,004 is a continuation-in-part of application No. PCT/US05/01556, filed on Jan. 21, 2005.
(60) Provisional application No. 60/538,847, filed on Jan. 23, 2004.

(51) Int. Cl. A61B 18/20; A61B 17/322

(52) U.S. Cl. CPC A61B 18/203; A61B 17/322
USPC 606/9; 606/131

(57) ABSTRACT

A method for reducing human or animal tissue includes making a plurality of excisions to collectively form a patch of tissue to be reduced, instead of making a single large excision in the patch. Thus, scarring after healing is less noticeable. The treated regions of tissue may be arranged such that a total area of all removed tissue segments taken in a direction perpendicular to an axis of the patch changes gradually along said axis. The removed regions of tissue may be navicular in shape, and the patches may be navicular in shape in the event an elongated excision is made. The removal of tissue regions can be realized by proper treatment methods such as incisions and laser treatment.

### RELATED APPLICATIONS

This application is a continuation-in-part of U.S. application Ser. No. 11/175,004 filed Jul. 5, 2005, which is a continuation-in-part of International Application No. PCT/US 05/01556 filed Jan. 21, 2005, which claims benefit of Provisional Application No. 60/538,847 filed Jan. 23, 2004. The disclosures of these applications are incorporated herein by reference.

### BACKGROUND

The present disclosure relates to techniques in the field of surgical instruments and methods for skin reduction or tightening, and more particularly, to a method and apparatus for reducing tissue to remove wrinkles and laxities in the tissue with minimal scarring after healing.

A number of methods have been used to tighten skin for the purpose of restoring a youthful appearance. Several of these methods are well known in the art and include dermabrasion, laser resurfacing and chemical peels. In these facial resurfacing processes, the epidermis is denuded to a certain depth by direct physical, chemical or thermal injury applied to the skin. The cutaneous injury induces a healing response, resulting in the deposition of a new skin matrix with improved appearance. Skin rejuvenation occurs by a proliferation of fibroblast activity, the action of inflammatory mediators, and a deposition of new collagen and other dermal matrix proteins. The object of these methods is to create a smoother and more attractive epidermal layer.

Using a different principal, U.S. Pat. No. 3,911,909 to Di Matteo discloses a device that applies pressure to the interior surface of the mouth and the adjacent exterior surface of the face for the purpose of mechanically flattening facial wrinkles. In a method described by U.S. Pat. No. 3,949,741 to Hofmann, pressure-sensitive adhesive film is applied to the skin, left for a few hours, and stripped off, removing with it a layer of dead epidermal cells. A number of patents describe various means by which collagen in the skin is caused to contract by heating with lasers or electromagnetic radiation. These processes create collagen scarring below the skin surface, which tightens the skin at the treated site. U.S. Pat. No. 6,241,753 to Knowlton describes a method by using electromagnetic radiation and U.S. Pat. No. 5,370,642 to Keller describes a method using laser energy. A noninvasive surgical method for tightening the skin is described in U.S. Pat. No. 5,713,375 to McAllister wherein a skin scratching tool with a plurality of generally parallel cutting blades is applied to the skin. The resulting parallel “scratches” in the skin heal and thereby cause the skin to tighten. Another method involves subcutaneous implanting of gold threads. The threads are implanted in sub-dermal space at the level of the derma inner edge and are aligned along and/or across wrinkles and skin folds. This method is said to induce collagen formation.

Within the category of surgery, it is generally known that plastic surgeons perform face-lifts and other types of rhytidectomies to tighten skin on the face, arms and other parts of the body. In a face-lift procedure, the surgeon begins an incision in the area of the temple hair, just above and in front of the ear, and then continues around the lobe, circling the ear before returning to the point of origin in the scalp. The skin is raised outward before the surgeon repositions and tightens the underlying muscle and connective tissue. Some fat may be removed, as well as excess skin. For men, the incision is aligned to accommodate the natural beard lines. In all cases, the incision is placed where it will fall in a natural crease of the skin for camouflage. One drawback resulting from placing an incision remote from the specific area of laxity is that the surgery stretches more skin than is necessary to reduce skin laxity or to reduce wrinkles. Such stretching reduces skin thickness and limits the number of times such procedures can be repeated. In addition, large scale pulling of the skin toward the hairlines can, in some cases, give the face an artificial look considered by many to be characteristic of apparent or repeated face-lifts. In addition, such procedures do not always resolve wrinkling in the mouth and chin areas. Moreover, such procedures do not lend themselves to resolving problems in other small areas of the body such as wrinkling at the junction of the thumb and forefinger.

In conventional skin treatment surgical practice, surgeons typically use a scalpel to remove skin abnormalities such as lesions or tumors. This is different from diagnostic skin sampling wherein biopsy punches are used to reduce reliance on the personal skill of the medical doctor. Such diagnostic punches are disclosed by U.S. Pat. No. 3,990,451 to Gibbs and U.S. Pat. No. 5,183,053 to Yeh et al. To avoid the unpleasant “dog ears” that may result from a circular or oval-shaped incision, for example, surgeons usually make a navicular-shaped incision (boat-shaped), around the lesion or tumor. However, these skin treatment techniques may not result in a satisfying aesthetic appearance if applied to the practice of cosmetic surgery since a single incision to reduce skin area for removing wrinkles or laxities may result in noticeable scarring after healing.

Therefore, an improved method is needed for reducing human and/or animal tissue, such as skin and/or muscle tissue, which method addresses the limitations of existing methods.

### SUMMARY

According to a first aspect of the present disclosure, a method for reducing tissue is provided, which results in less noticeable scarring.

In one embodiment the method may comprise forming a two-dimensional array of excisions in the tissue, the array having first and second ends, each of the excisions of the array defining an area where a portion of the tissue has been removed from the tissue; wherein at any point between the first and second ends of the array, the total measurement across all excisions at the point gradually increases from the first end of the array to a maximum value between the first and second ends of the array and then gradually decreases moving toward the second end of the array.

In some embodiments, the array has a width that gradually increases moving from the first end thereof toward the second end thereof, the width gradually increasing to a maximum between the first and second ends of the array and then gradually decreasing moving toward the second end thereof.

In some embodiments, the array has a length which is greater than the second width.

In some embodiments, the array has a substantially navicular shape or a substantially elliptical shape.

In some embodiments, at least one of the excisions has a substantially navicular shape or a substantially elliptical shape.

In some embodiments, the array extends in a direction of least tissue tension of the tissue.

In some embodiments, the excisions of the array are formed one at a time.

In some embodiments, the forming is performed by a scalpel, a punch, a laser, or any combination thereof.

In some embodiments, the excisions of the array are formed substantially at the same time.

In some embodiments, the forming is performed by a laser and wherein the forming comprises moving the laser from one region of the tissue to another region of the tissue.

In some embodiments, the laser comprises a narrow beam laser.

In some embodiments, the forming further comprises turning on the laser after it arrives at a region of the tissue; and turning off the laser during movement from one region of the tissue to another.

In some embodiments, two or more of the excisions are formed substantially at the same time. In such embodiments, the forming may be performed by a laser. In such embodiments, the laser may be a wide beam laser and wherein the forming comprises moving the laser from a first region of the tissue to at least a second region of the tissue. In such embodiments, the forming may further comprise turning on the laser after it arrives at the first region of the tissue and turning off the laser during movement from the first regions of the tissue to the at least second region of the tissue.

In some embodiments, the method further comprises closing the excisions of the array.

In some embodiments, the closing is performed by suturing, with an adhesive, by laser welding, or any combination thereof.

In some embodiments, at least two of the excisions have shapes, which are substantially different from one another.

In some embodiments, the total measurement across all excisions at the point gradually increases from the first end of the array to a maximum value between the first and second ends of the array in a monotonic or non-monotonic manner and then gradually decreases in a monotonic or non-monotonic manner moving toward the second end of the array.

According to a second aspect of the present disclosure, an apparatus for reducing tissue is provided, which comprises an array of punches for incising a patch of tissue, the punches being arranged to collectively form an operational region that matches the patch of tissue to be treated.

The punches may be dimensioned and arranged such that a total area of all incised tissue segments taken in a direction perpendicular to an axis of the patch changes gradually along the patch axis.

The operational region and the patch may have a substantially elliptical or navicular shape. Each punch may have a cutting edge that is substantially navicular in shape.

### BRIEF DESCRIPTION OF THE DRAWINGS

In the drawings, like elements may be identified by like reference characters.

FIG. 1a schematically illustrates a plan view of a plurality of excisions made in tissue according to an embodiment of the disclosure.

FIG. 1b schematically illustrates the plurality of excisions illustrated in FIG. 1b, after closure thereof

FIG. 1c schematically illustrates the plurality of excisions illustrated in FIG. 1a, arranged according to an “area rule,” which collectively form a substantially diamond-shaped area of tissue to be reduced.

FIG. 2 illustrates a plurality of excisions made according to another embodiment of the disclosure arranged according the “area rule,” which collectively form a substantially navicular-shaped area of tissue that will be reduced.

FIG. 3 illustrates a plurality of differently-shaped excisions made according to a further embodiment of the disclosure arranged according to the “area rule,” which collectively form a substantially elliptical-shaped area of tissue to be reduced.

FIG. 4 illustrates an embodiment of a punch array according to the present disclosure.

FIG. 5 is a sectional view illustrating an embodiment of apparatus according to the present disclosure which incorporates the punch array shown in FIG. 4.

FIG. 5a partially illustrates an alternative embodiment of the apparatus in FIG. 5.

FIG. 6 illustrates another embodiment of a punch array according to the present disclosure.

FIG. 7 illustrates an embodiment of the punch.

FIG. 8 illustrates an embodiment in which tissue is reduced using a laser and a mask.

FIG. 9 schematically illustrates a plan view of a plurality of excisions made in tissue according to another embodiment of the disclosure.

FIG. 10 illustrates another embodiment in which tissue is reduced using a laser and a mask.

### DETAILED DESCRIPTION

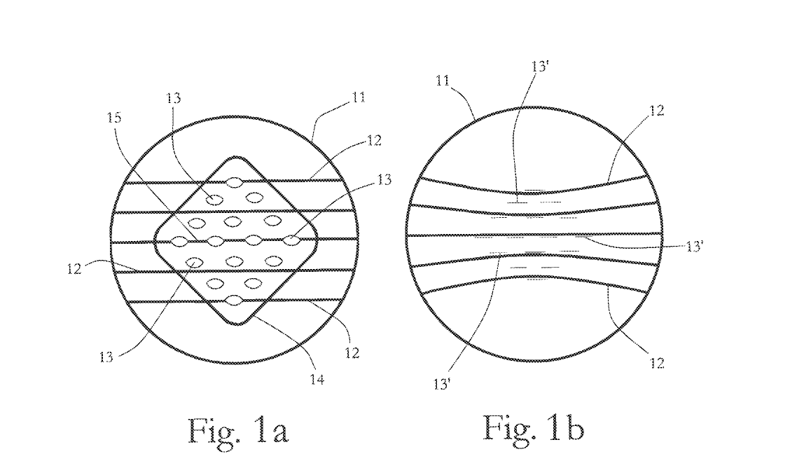

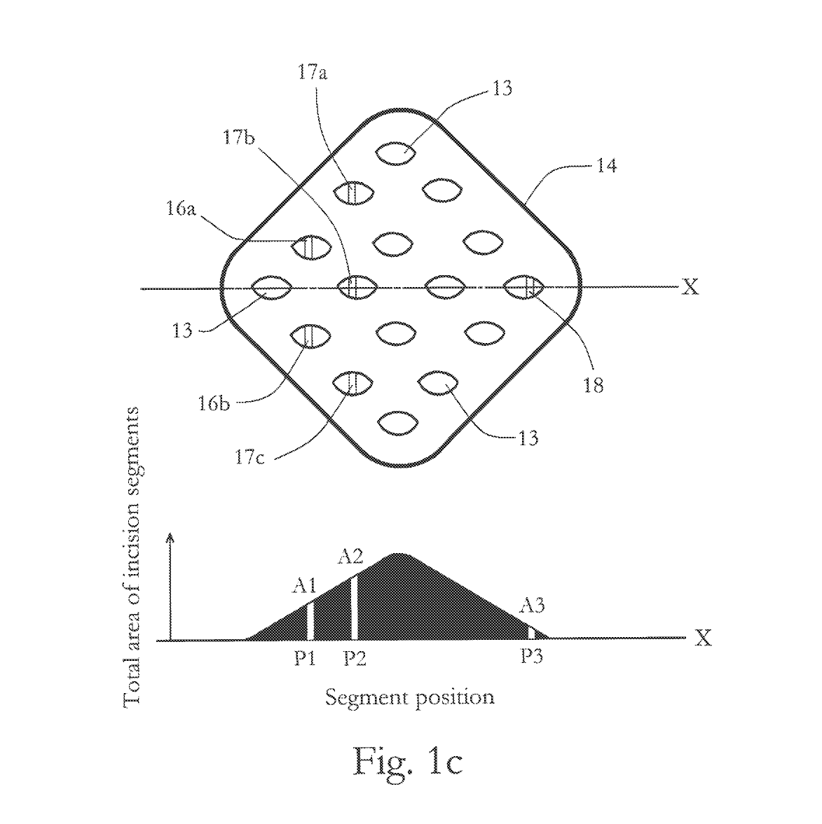

FIG. 1a is a plan view representation of a circular section of human or animal tissue 11, such as human skin, with parallel prereduction orientation lines 12 and an array of excisions 13 formed in the tissue. The excisions 13 may be formed using a scalpel, a punch, a laser, or any combination thereof. The array of excisions 13 collectively form an area or patch 14 of tissue that is to be tightened or treated. FIG. 1b is a plan view representation of the circular section of tissue 11 shown in FIG. 1a after closure of the excisions 13. The closed excisions are shown as short lines 13′ in FIG. 1b, which are usually unnoticeable when the excisions 13 are very small.

The array of excisions 13 may be arranged according to an “area rule” wherein the total area of excision segments taken in a direction perpendicular to an axis 15 of the tissue patch 14 changes gradually along the axis 15. It is understood that such gradual changes include incremental increases, stasis (if any) and incremental decreases. Preferably, no discontinuity exists in the change.

The “area rule” is now explained in more detail with reference to FIG. 1c, which is a plan view of the array of excisions shown in FIG. 1a with an associated relative area graphical representation that represents a total area of excision segments along an axis “X” of the patch 14. The total areas and corresponding excision segments at three positions P1, P2 and P3 along the axis “X” are exemplarily shown. Specifically, total area A1 at position P1 is a sum of areas of excision segments 16a and 16b which are taken at the position P1 in a direction perpendicular to the axis “X”. All the segments are considered to have the same width, which is a predetermined length of the axis “X”. Similarly, total area A2 is a sum of areas of excision segments 17a, 17b and 17c taken at the position P2, and total area A3 is the area of the excision segment 18 taken at the position P3. According to the “area rule”, the total area of the excision segments taken in a direction perpendicular to the axis “X” changes gradually along the axis “X”, as illustrated in the area graphical representation in FIG. 1c. In other words, the collective excision area change along the axis “X” of the array maintains a gradual and continuous increase, stasis (if any) and decrease in collective excision area perpendicular to the axis “X”, preferably without discontinuities in incremental area change. Since all the segments have the same width, the “area rule” may also be interpreted as that the sum of the heights of all the segments corresponding to a point on the axis “X” changes gradually along the axis “X”.

Observing this “area rule” when designing surgical excision arrays will provide a continuous and balanced tightening of tissue on closing the array excisions, equivalent to having performed one large area rule excision such as in a single substantially elliptical or navicular excision performed by a scalpel, a biopsy punch, or laser, but without one large noticeable scar. The closed excisions 13′ collectively pull the tissue on either side of the axis “X” of the reduced patch in a gradual and uniform manner as if in a single large navicular excision. The scar pattern in FIG. 1b demonstrates this concept.

The axis “X” of the patch 14 that is to be reduced may be oriented parallel to the lines of least tissue tension of the patch 14, such as lines perpendicular to the long axis of an arm.

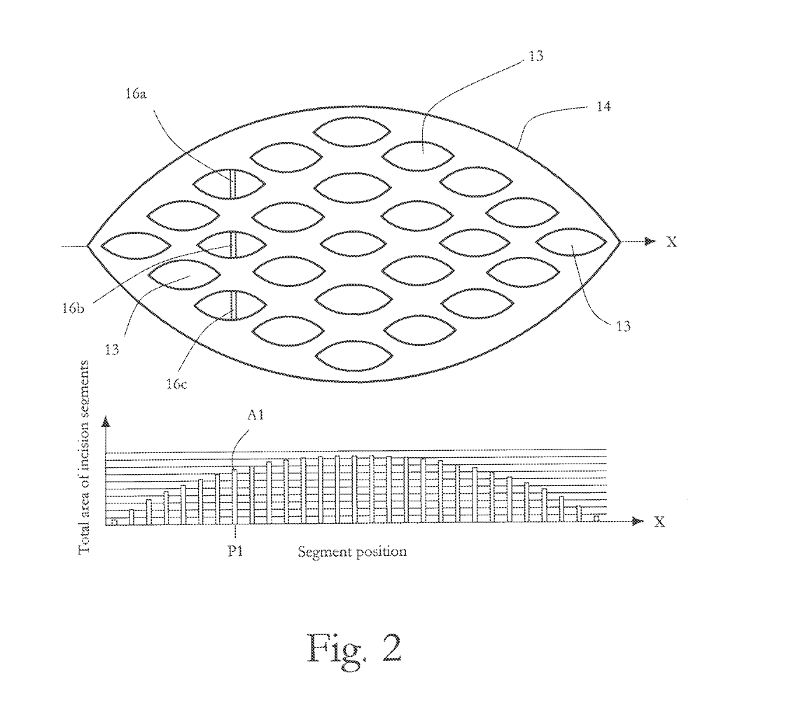

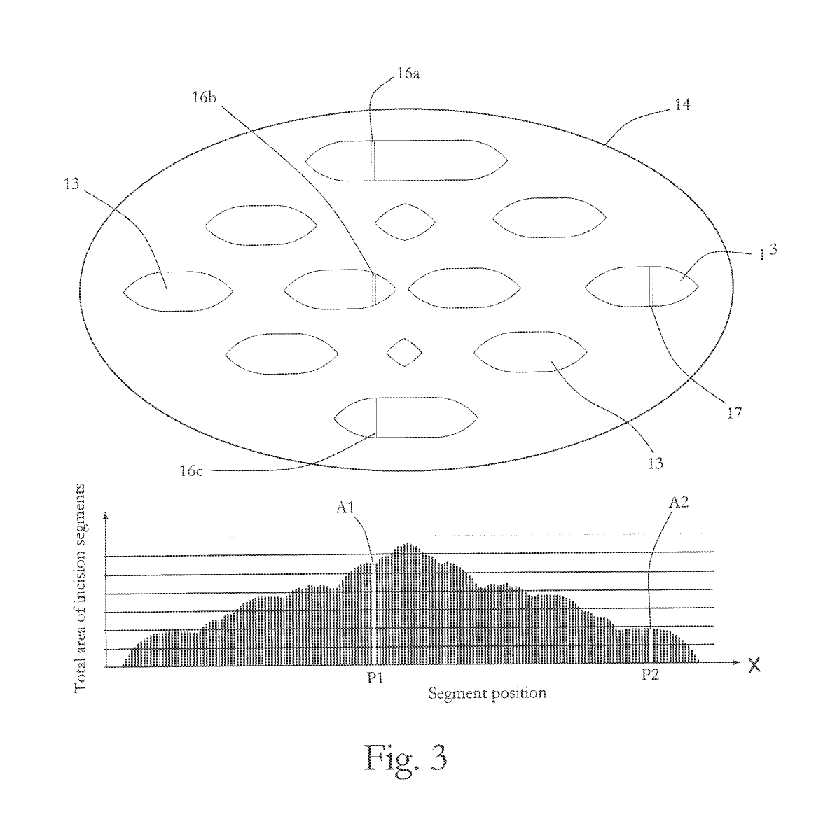

The tissue patch 14 to be treated or reduced, which is collectively formed by the plurality of excisions 13 arranged following the “area rule”, may be of a substantially navicular shape (FIG. 2), a substantially elliptical shape (FIG. 3), a substantially circular shape, a substantially rectangular or elongated shape (such in a scalp reduction) or other proper shapes. The excisions 13 are may be substantially navicular in shape to avoid “dog ears”, with each excision being either substantially identical in size and navicular in shape as shown in FIG. 2, or with the excisions having substantially different sizes and shapes as shown in FIG. 3.

As described earlier, FIG. 2 illustrates an embodiment comprising a plurality of excisions 13, which are substantially identical in size and substantially navicular in shape. The excisions 13 in this embodiment will reduce a substantially navicular-shaped patch of tissue 14. The excisions 13 are arranged according to the “area rule” as explained above, and the total area of excision segments gradually increases and decreases along the axis “X” which extends through the two ends of the patch 14, as illustrated in the area graphical representation. For example, a total area Al at position P1 represents the sum of areas of the excision segments 16a, 16b and 16c taken at the position P1 in a direction perpendicular to the axis “X”.

FIG. 3 illustrates an embodiment in which a substantially elliptical-shape patch 14 of tissue is collectively formed by a plurality of different navicular-like shaped excisions 13. Although the excisions 13 are different, the total area of excision segments still follow the “area rule” as shown in the area graphical representation in FIG. 3. This can be done by properly selecting the dimensions, shapes and locations of the excisions 13. The computer software can be written to assist in determining the size, shape and placement of the excisions 13 for a given tissue patch 14 to be reduced. It is noted that exemplary total areas A1 and A2 at two positions P1, P2 and their associated excision segments 16a, 16b, 16c and 17 are illustrated in FIG. 3.

To make scar patterns less noticeable, the axes of the excisions 13 which extend across the two ends of the substantially navicular shapes may be arranged more or less randomly unparallel to the axis “X” of the tissue patch 14. In choosing the degree of variation from the axis “X”, the area rule may be followed since the excisions 13 will be closed by moving tissue on the lateral sides of the array substantially perpendicular to and toward the axis “X”. For arrays that require curved axes, e.g., extremely long arrays relative to their width, the axis “X” can curve to conform to tissue reduction requirements and lines of least tissue tension. An example of a curved array that would not be considered long is an overall circular array with excision axes that form a curve to conform to, for example, wrinkle areas in the skin around the mouth or eyes.

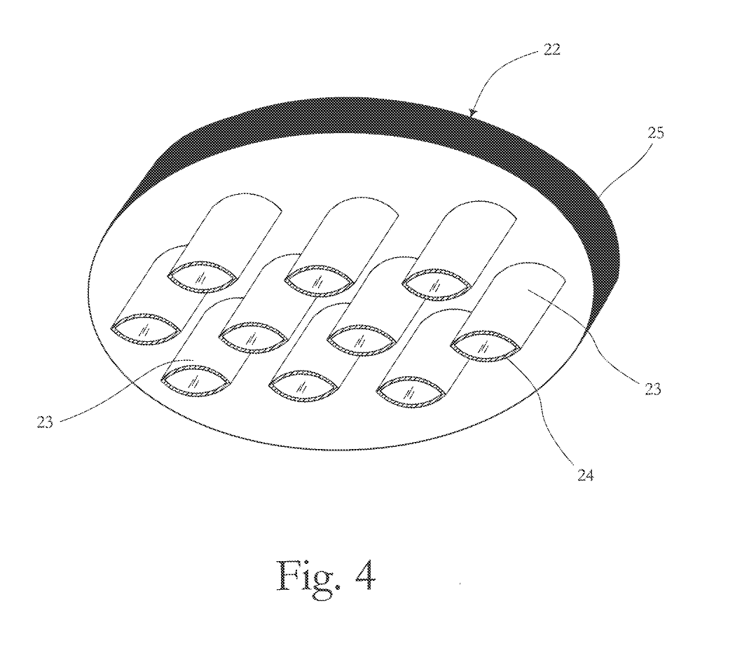

The method of the present disclosure may be carried out by a punch array 22, which simultaneously makes a plurality of incisions in the tissue. Each of the incisions encircles a portion of the tissue, which when removed defines an excision. Therefore, the punch array 22 can be used for forming the array of excisions 13 mentioned earlier, which collectively form the tissue patch 14 to be reduced. As illustrated in FIG. 4, a punch array 22 basically comprises an array of punches 23 collectively forming an operational region that matches the tissue patch 14 to be reduced. An end of each punch 23 is provided with a cutting edge 24, and the other end is attached to a platform 25.

The cutting edge 24 of each punch 23 is sized and shaped to be suitable to make a corresponding excision 13 within the tissue patch 14, and the punches 23 can be designed and positioned to meet the requirements of each specific type of tissue reduction. For example, a scalp reduction might employ a two inch linear array of two rows of navicular punches using a flexible array support means to permit adaptation to the non-planer skin surfaces of the scalp, whereas a micro-face-lift surgical punch array might employ a configuration comprising a one-half inch elliptical array of five parallel rows of elliptical punches with different lumen sizes varying from one to three millimeters.

For large tissue reductions, the method of the present disclosure can be implemented by using means to identify placement, shape and orientation of the excisions 13, such as a template or other marking means, and forming the excisions 13 by hand using a scalpel or surgical punch to cut or incise the tissue. It is also contemplated to use machines to make excisions with lasers or other cutting means to perform a skin reduction consistent with the methods of the present disclosure.

In skin tissue reductions, it is preferred that the depth of the dermal layer is determined before applying the punch array 22 or other excision forming device to make the excisions 13. Electronic or other means for determining dermal layer depth would make both machine and manual array excisions 13 more precise and safer by preventing incising delicate structures below the dermal layer. Also, excision position guides, e.g., rings in the shape of the excision 13 with adhesive for temporary attachment to the skin at predetermined excision sites, can be placed in an array to ensure proper placement of surgical punches or incisions to ensure precise reapplication of surgical punches or hand incisions if the initial incision did not cut completely through the dermal layer.

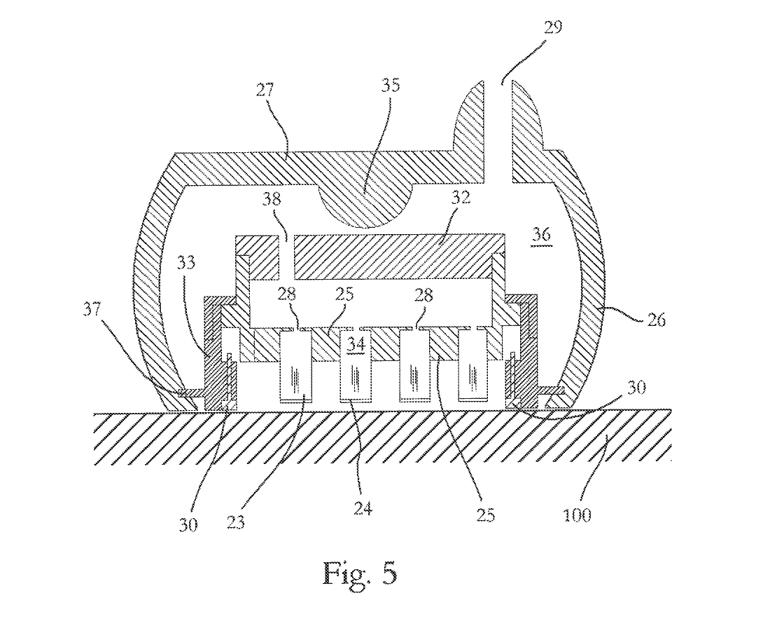

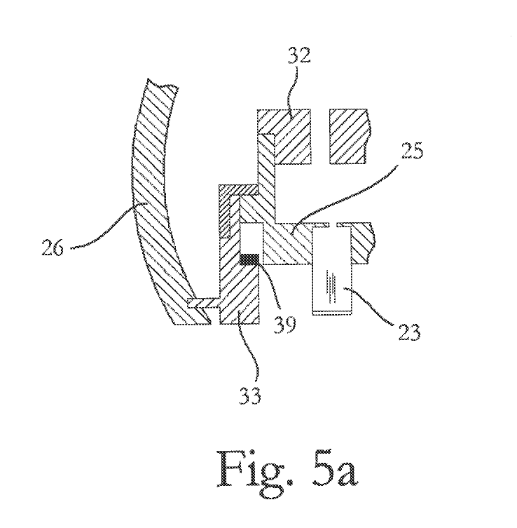

An embodiment of surgical apparatus incorporating the punch array 22 according to the present disclosure is illustrated in FIG. 5. Although punches 23 can be replaced individually, the punches 23 can be changed by removing cylinder 33 from a flexible housing 26 by disengaging member 37 and replacing the used cylinder 33 with a new cylinder 33. Each punch 23 is a hollow tube with a lumen 34. A cutting edge 24 is provided at the free end of each punch 23, either being formed as an integral part of the punch 23 or is attached to the free end of the punch 23.

The platform 25 is held and accommodated inside the cylinder 33 and is slidable along an inner surface of the cylinder 33. The cylinder 33 is secured to the flexible drum-like housing 26 by the horizontal member 37.

The drum-like housing 26 has an open bottom, which allows the punches 23 to move therethrough toward the tissue 100. The drum-like housing 26 has a top 27 having a hemispherical bulge 35 under its undersurface. The top 27 (or the whole housing 26) is may be made of a resilient material such that when a force is applied on the top 27, the top 27 deforms and falls in. This brings the bulge 35 into contact with a cap 32 of the platform 25, and drives the cap 32 and therefore the platform 25 and the punches 23 to slide downward inside the cylinder 33 towards the tissue 100, whereby cutting into the tissue 100 for making the desired excisions 13. When the force is removed, the resilient top 27 returns to its initial state. A biasing mechanism, such as a restoring spring (not shown), may be provided to return the platform 25 as well as the punches 23 back to their initial state as shown in FIG. 5 when the force applied on the top 27 is removed.

A movement-limiting mechanism may be provided to limit the sliding movement of platform 25 to a predetermined amount, whereby limiting the depth that the punches 23 cut into the tissue 100. For example, as shown in FIG. 5, a stop ring 30 may be provided to limit the amount of the downward displacement of the platform 25. The stop ring 30 may be adjustably mounted to the cylinder 33 so as to adjust the amount of the movement of the platform as desired. Alternatively, the stop ring 30 can be replaced by one or more bolts mounted to the cylinder 33 to limit the vertical movement of the platform 25 to a desired amount, and the bolts may be adjustable vertically.

Alternatively, as illustrated In FIG. 5a, one or more washer-like rings 39 may be placed between an undersurface of the platform 25 and an inner shoulder of the cylinder 33 so as to limit the vertical movement of the platform 25 to a desired amount.

As shown in FIG. 5, a relief hole 28 may be provided at the top of each punch 23 so that the air inside the lumen 34 can escape from the relief hole 28 when the punch 23 cuts into the tissue 100, whereby avoiding forming a pressure inside the lumen 34 above the tissue plug that enters the lumen 34.

Suction may be applied to the lumen 34 through the relief hole 28 to hold the tissue plug inside the lumen 34 so it can be removed together with the punch 23 when the punch 23 is withdrawn from the tissue, or to help detaching the tissue plug from the subdermal tissue. The suction can be applied from a vent 29 provided on the top 27 of the housing 26, and communicates with each punch 23 via a port 38 provided on the cap 32 of the platform 25 and each relief hole 28. Besides, as an alternative to applying an external force on the top 27 to drive the cap 32, by reducing pressure in housing 26, outside pressure deforms housing 26 and hemispherical bulge 35 presses against cap 32, thereby driving the cutting array of punches 23 into the tissue 100.

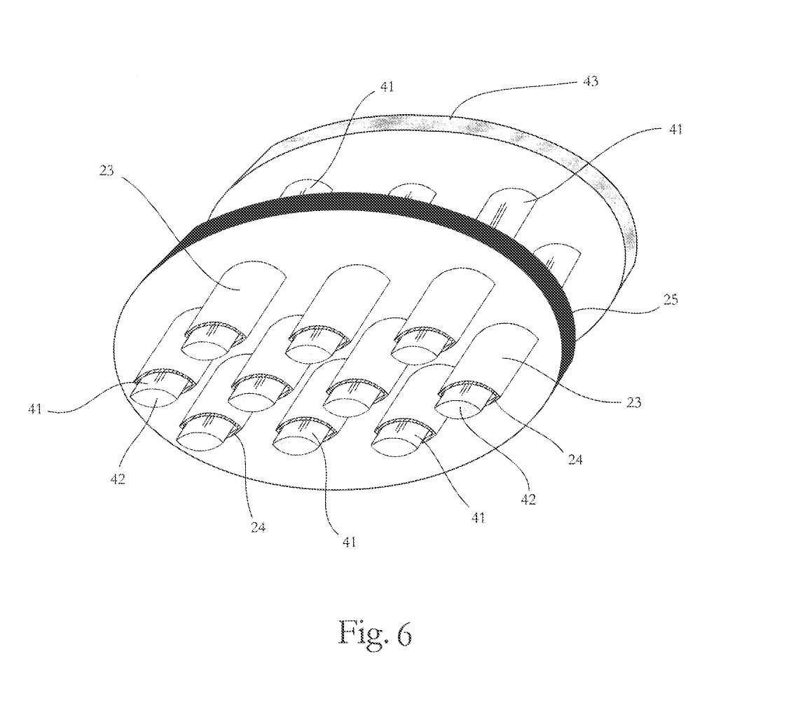

Alternatively, as illustrated in FIG. 6, an in-line array of gripping rods 41 are arranged to pass through their counterpart surgical punches 23. The leading surfaces of the gripping rods 41 are coated with a tissue adhesive 42 that is intended to contact the tissue 100 such that the incised tissue plugs are gripped by the adhesive 42 and are forcibly removed as the array of rods 41 are removed after incision.

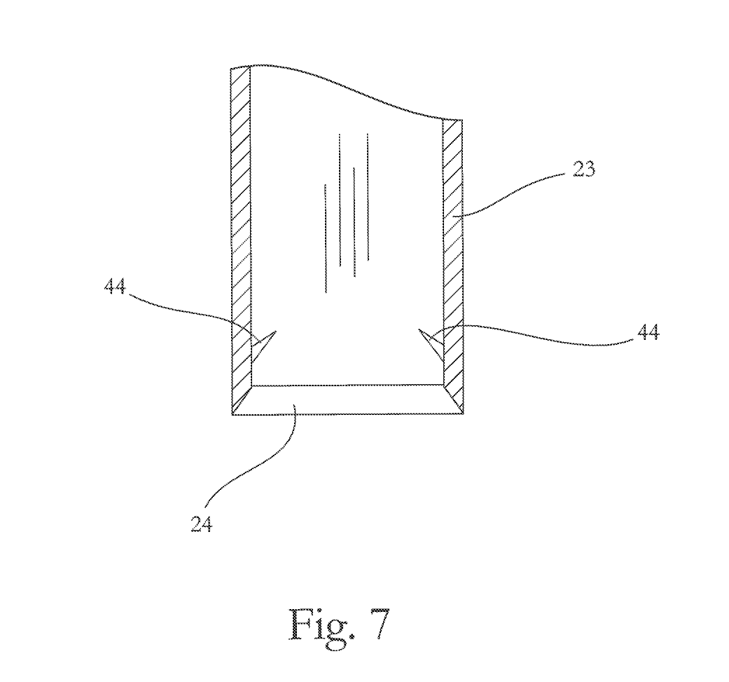

Alternative or in addition to the adhesive rods 41, one or more upward barbs 44 are provided on the inner walls of the surgical punches 23, as shown in FIG. 7. The barbs 44 secure the tissue plugs within the surgical punches 23 and forcibly remove the plugs as the surgical array is removed. Alternatively, a spear-like barb or equivalent (not shown) can be attached to the end of rods 41 to mechanically engage and withdraw tissue plugs formed by incising with the punches 23.

A skin incision operation incorporating the teaching of the present disclosure is described further below. When the array of punches 23 are applied to the skin 100, multiple precise excisions 13 are made through the dermal layer. If the punches 23 have lumens 34 measuring a few millimeters, the skin plugs created by the punches 23 will be withdrawn with the array of punches 23. If the lumens 34 are larger and barbs 44 or adhesive rods 41 are not employed to hold the skin plugs within the punches 23 as they are withdrawn, the in situ plugs can be removed surgically or by applying suction to the array of punches 23 through the relief holes 28 when the incisions are being made, such that incised plugs of skin are drawn into and retained in the lumens 34 of the surgical punches 23 as they are removed. If incised plugs still remain attached to the sub-dermal tissue, other means for removal should be employed, such as with a pin or tweezers and possibly scissors to cut the plugs free from the fat layer. In addition, means for reciprocating the surgical punches 23 or punch array 22 to enhance its cutting action can be attached to means (e.g., the housing 26) used by the surgeon to hold the surgical cutting device during its use.

In a typical skin reduction application, after the patch 14 of skin to be reduced is selected, the skin is cleansed and anesthetized, e.g., with two percent lidocaine with epinephrine. In determining how to orient the surgical punch array 22, the lines of least skin tension should be identified. For example, on the arm these lines run perpendicular to the long axis of the arm. The axes of surgical punches will normally be oriented parallel to the lines of least skin tension. To apply the surgical punch array 22 to make an array of excisions 13, skin surrounding the excision site (i.e., the selected patch) can be stretched with the thumb and index finger perpendicular to the lines of least skin tension, e.g., along the long axis of the arm. Once the punch array 22 has penetrated the dermis and begins to penetrate the subcutaneous fat layer, penetration should be stopped and the array removed. When using a surgical punch array 22 on areas where the skin is thin, such as the face, neck or distal extremities, it is possible to damage arteries, nerves and veins below the skin. Most surgeons can identify when a hand-held punch penetrates the dermis because a “give” can be felt. With a surgical punch array 22, feeling a “give” might not be possible. Therefore, methods for determining skin thickness and means for presetting the depth of penetration may be employed when using a surgical punch array 22 on areas like the face, neck or distal extremities.

Upon withdrawal of the surgical punch array 22 from the skin 100 there may be a tendency of the cut skin to remain in place. Suction forcing the tissue into the surgical punch 23 is recommended. Alternatively or in addition to the use of suction, an upward pointing barb or hook 44 can be situated inside the punch 23 to engage and retain the skin. It is further recommended that relief holes 28 be placed in the upper portion of each surgical punch 23 to allow air trapped in the punch 23 during incision to escape. When suction is used, these same relief holes 28 provide a passage through which the suction source can communicate with the skin plugs to draw them into the surgical punch array 22 for extraction. In the event plugs of skin are not removed as the surgical incision array 22 is removed, as would be expected for large surgical incision arrays, these plugs of skin may be removed with appropriate instruments while being careful to prevent skin from dropping down and lodging under the dermis layer. Scissors can be used to cut the skin below the dermis to free it from subcutaneous tissues. After the skin plugs are removed, array incision dimensions in the direction of the initial skin stretching will reduce as the skin is released and relaxes to its original state. This process results in a partial closing of the excisions 13. For larger surgical punch excisions, the excisions may be closed with an appropriate number of sutures using, e.g., 5-0 or 6-0 nylon. The suture generally creates good hemostasis, and antibiotic ointment and a bandage can be applied. For smaller excisions, a cyanoacrylate tissue adhesive can be applied instead of sutures. Cyanoacrylate adhesive forms a strong bond across apposed excision edges, allowing normal healing to occur below. These adhesives are marketed to replace sutures that are 5-0 or smaller in diameter for incisional and other repair. This adhesive provides a flexible water-resistant protective coating, eliminates the need for suture removal, and, when very small surgical punches 23 are used, adhesive closure might be the only practical closure method. The long-term cosmetic outcome with cyanoacrylate adhesives is comparable to that of traditional methods of repair. Smaller excisions 13 can often be cleansed with an antibacterial compound and flushed with sterile saline solution before closure. The edges of the excision must be approximated manually and evenly. If there is uncertainty about whether this can be done, the excision should probably be sutured instead. Once the edges have been approximated, adhesive is applied to the apposed excision edges. After applying adhesive across the excision edges and holding the edges together for at least thirty seconds before releasing, more adhesive should be applied in an oval pattern around the excision to encompass a greater surface area on the skin. This adds greater strength to the excision closure. At least three layers should be applied to ensure optimal strength and no added coverings are needed. The first layer of adhesive reaches maximal strength within two and one-half minutes. The adhesive will spontaneously peel off in five to ten days. The excisions 13 may also be closed using a laser welding technique, such as disclosed in U.S. Pat. No. 5,620,435 to Belkin et al., the entire disclosure of which is incorporated herein by reference.

With respect to skin applications, the use of the apparatus of the present disclosure can be repeated, e.g., on an outpatient basis until the desired amount and direction of skin reduction is achieved. If multiple visits are employed, the results of previous procedures can be adjusted with each new visit by choosing the precise size and placement of subsequent punch array applications to perfect the skin reduction.

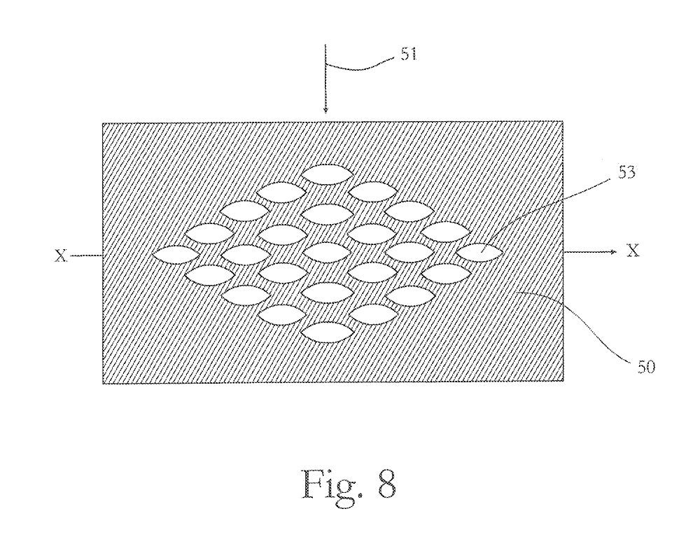

As an alternative to mechanical excisions by, e.g., the array of punches, the tissue reduction may be realized by laser treatment. As illustrated in FIG. 8, a mask 50 may be provided to cover the patch of tissue to be reduced by a laser 51. The mask 50, which is made of a sheet or panel of material capable of preventing the laser 51 from passing through, may comprise a plurality of holes 53 that allow the laser beam 51 to pass through. Each hole 53 exposes a region of tissue to the laser 51 to be reduced by the laser 51. The laser 51 vaporizes the regions of tissue exposed by the holes 53 on the mask 50. The duration of laser exposure should be sufficient to vaporize the tissue (e.g., dermal layer), but insufficient to vaporize sub-dermal or other tissue.

The holes 53 may be dimensioned and arranged to follow the “area rule”, i.e., a total area of all removed or treated tissue segments taken in a direction perpendicular to an axis “X” of the patch with a predetermined width along the axis “X” varying gradually along said axis “X”. In one embodiment, the holes 53 are navicular in shape.

The laser 51 may comprise one or more wide area beams each covering one or more of the holes 53. Because of the protection of the mask 50, only the regions defined by the holes 53 are treated by the laser beams.

Some or all of the wide area beams may stay statically above the relevant hole or holes 53, or may move around so that one wide area beam may treat different regions one after another.

As an alternative, the laser 51 may comprise a single narrow beam that moves from one region to another in a predetermined pattern that is equivalent to the pattern of regions of tissue exposed by the laser mask 50 described above. The beam can be turned off when it is moving, and turned on after it is moved to a desired position for treating a targeted region. Thus, the single beam may treat all the tissue regions. In addition, the mask 50 may be omitted if the narrow beam has a cross-sectional shape that matches each region.

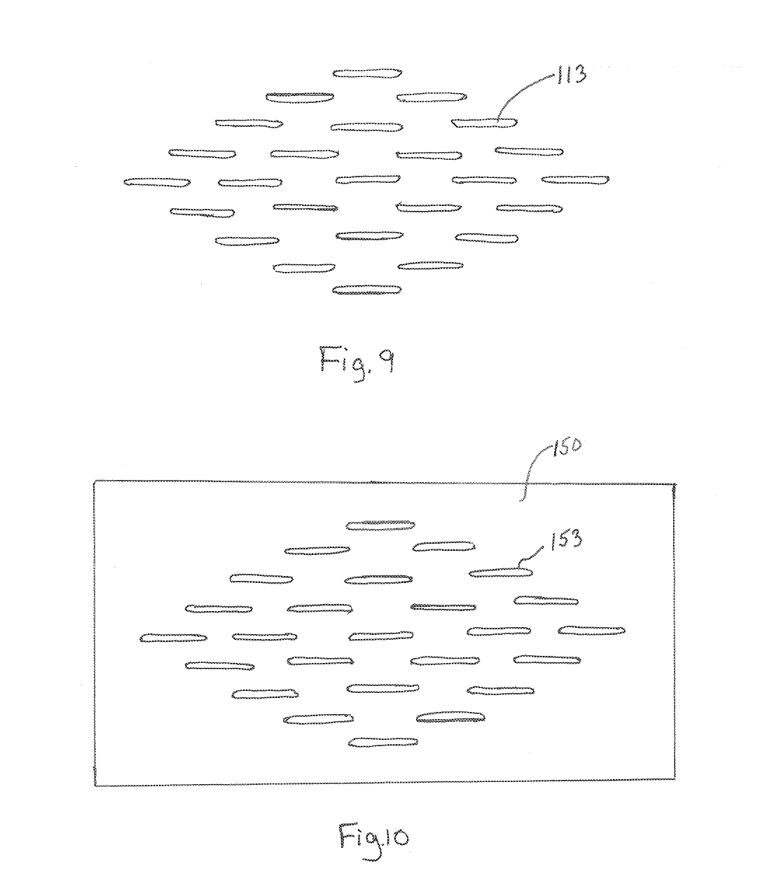

In another embodiment, as illustrated in FIG. 9, instead of treating substantially navicular or elliptical regions of tissue, a laser may be used to perform narrow line excisions, which may be measured in millimeters and smaller. As illustrated in FIG. 9, the laser may make multiple, substantially parallel narrow line excisions 113 of the same length, which collectively form the patch of tissue to be treated. In other embodiments, the laser may make multiple, narrow line excisions 113 where one or more of the excisions 113 are not substantially parallel to one another. In such embodiments, one or more of the narrow line excisions 113 may be the same length or different lengths. Compared to treating the patch with a single large incision, multiple narrow line excisions 113 reduce the appearance of scarring even without following the “area rule”. In addition, with narrow line excisions 113, the “dog ear” effect experienced with wider excisions would not be significant. The laser may be configured to generate a single beam having a diameter that makes one narrow line excision 113 at a time. In other embodiments, the laser may be configured to generate multiple beams each having a diameter that makes one narrow line excision so that multiple narrow line excisions 113 can be made simultaneously.

As illustrated in FIG. 10, a mask 150 having an array of the holes 153 each having a narrow line shape, may be used with a laser to generate the narrow line excisions 113 illustrated in FIG. 9. In such embodiments, the laser may be configured to generate a single beam having a diameter that allows one or more narrow line excisions 113 to be made at a time in a sequential manner using the mask 150. In further embodiments, the laser may be configured to generate a single beam or multiple beams that have a diameter or diameters that allow multiple or all the narrow line excisions 113 to be made simultaneously using the mask 150.

The beginning and end areas of the line excision array may follow the gradual transition “area rule”, while the substantially parallel and continuous portion of the excision array between the beginning and end areas would experience no combined excision area transition. The narrow line excisions may be spaced apart sufficiently.

When applied to excising skin tissue, it is necessary to control the depth as well as the pattern of laser cutting. Although Fitzpatrick scale of skin types can be used for a given laser to estimate penetration levels for different shades of skin color, an alternative would be to use the equivalent of an ultrasound scanner designed to measure skin thickness and subsurface structures. It is likely that cut depth testing with a scanning ultrasound device, such as those used to analyze decubitus ulcers, would be useful in determining the settings for excising tissue with a motion controlled narrow beam laser as well as testing the cut depth after each exposure of wide area lasers that can be used multiple times to excise tissue on all or part of a mask described above.

Although the foregoing has described the preferred embodiments of the present disclosure, it shall be understood that, without departing from the spirit of the present disclosure, numerous variations, adaptations and modifications are available to a person with ordinary skill in the art. For example, instead of a simultaneous operation by the punch array, the plurality of excisions can be made individually one by one by a small punch or other apparatus such as described in Publication No. US 2010/0331867 to Giovannoli and Publication No. US 2013/0041385 to Giovannoli, the entire disclosures of which are incorporated herein by reference. Additionally, after incising the tissue via a punch or other incising means, the plug of tissue may be excised by vaporizing the plug of tissue. Moreover, the excising of tissue may be carried out using other suitable methods tissue removal methods. Therefore, the scope of invention is intended to be solely defined by the accompanying claims.

What is claimed is:

1. A method for reducing human or animal tissue, the method comprising:
forming a two-dimensional array of excisions in the tissue, the array having first and second ends, each of the excisions of the array defining an area where a portion of the tissue has been removed from the tissue;
wherein at any point between the first and second ends of the array, the total measurement across all excisions at the point gradually increases from the first end of the array to a maximum value between the first and second ends of the array and then gradually decreases moving toward the second end of the array.

2. The method of claim 1, wherein the array has a width that gradually increases moving from the first end thereof toward the second end thereof, the width gradually increasing to a maximum between the first and second ends of the array and then gradually decreasing moving toward the second end thereof.

3. The method of claim 2, wherein the array has a length which is greater than the second width.

4. The method of claim 1, wherein the array has a substantially navicular shape or a substantially elliptical shape.

5. The method of claim 1, wherein at least one of the excisions has a substantially navicular shape or a substantially elliptical shape.

6. The method of claim 1, wherein the array extends in a direction of least tissue tension of the tissue.

7. The method of claim 1, wherein one or more the excisions of the array are formed sequentially.

8. The method of claim 7, wherein the forming is performed by a scalpel, a punch, a laser, or any combination thereof.

9. The method of claim 1, wherein one or more the excisions of the array are formed substantially at the same time.

10. The method of claim 9, wherein the forming is performed by a scalpel, punch, a laser or any combination thereof.

11. The method of claim 1, wherein at least one of the excisions has a narrow line shape.

12. The method of claim 11, wherein the excisions are formed sequentially.

13. The method of claim 12, wherein the forming is performed by a laser.

14. The method of claim 12, wherein the forming comprises moving the laser from one region of the tissue to another region of the tissue.

15. The method of claim 14, wherein the laser comprises a narrow beam laser.

16. The method of claim 14, wherein the forming further comprises:
turning on the laser after it arrives at each region of the tissue; and
turning off the laser during movement from one of the regions of the tissue to another.

17. The method of claim 11, wherein two or more of the excisions are formed substantially at the same time.

18. The method of claim 17, wherein the forming is performed by a laser.

19. The method of claim 17, wherein the forming is performed by a wide beam laser and wherein the forming comprises moving the laser from a first region of the tissue to at least a second region of the tissue.

20. The method of claim 19, wherein the forming further comprises:
turning on the laser after it arrives at the first region of the tissue and the at least second region of tissue; and
turning off the laser during movement from the first the region of the tissue to the at least second region of the tissue.

21. The method of claim 1, further comprising closing the excisions of the array.

22. The method of claim 21, wherein the closing is performed by suturing, with an adhesive, by laser welding, or any combination thereof.

23. The method of claim 1, wherein at least two of the excisions have shapes, which are substantially different from one another.

24. The method of claim 1, wherein the total measurement across all excisions at the point gradually increases from the first end of the array to a maximum value between the first and second ends of the array in a monotonic or non-monotonic manner and then gradually decreases in a monotonic or non-monotonic manner moving toward the second end of the array.
